(19) United States
(12) Patent Application Publication
(10) Pub. No.: US 2019/0223402 A1
(43) Pub. Date: Jul. 25, 2019

(54) EGGPLANT PRODUCING SEEDS WITH A NOVEL COLOR

(71) Applicant: RIJK ZWAAN ZAADTEELT EN ZAADHANDEL B.V., De Lier (NL)

(72) Inventor: Johannes Henricus Jacobus Van Den Enden, De Lier (NL)

(21) Appl. No.: 16/245,409

(22) Filed: Jan. 11, 2019

Related U.S. Application Data
(63) Continuation-in-part of application No. PCT/EP2016/066809, filed on Jul. 14, 2016.

(51) Int. Cl. A01H 6/82; A01H 5/08

(52) U.S. Cl. CPC A01H 6/826; A01H 5/08

(57) ABSTRACT

The present invention relates to an eggplant plant, in particular a Solanum melongena plant comprising a QTL, which leads to the plant producing seeds that have a novel color, wherein said QTL is obtainable by introgression from a plant grown from seed of which a representative sample was deposited with the NCIMB under deposit number NCIMB 42507. The invention also relates to seeds and other propagation material of the plant and to its progeny as well as to food products that comprise the consumable parts of the plant.

### RELATED APPLICATIONS AND INCORPORATION BY REFERENCE

This application is a continuation-in-part application of international patent application Serial No. PCT/EP2016/066809 filed 14 Jul. 2016, which published as PCT Publication No. WO 2018/010803 on 18 Jan. 2018.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE STATEMENT

The instant application contains a Sequence Listing which has been filed electronically in ASCII format and is hereby incorporated by reference in its entirety. Said ASCII copy, created on Jul. 14, 2016, is named Sequence Listing.txt and is 8 bytes in size.

### FIELD OF THE INVENTION

The present invention relates to a novel eggplant (Solanum melongena L.) which produces seeds with a novel seed color. The invention further relates to the seeds and progeny of such plants and to propagation material for obtaining such plants. Furthermore the invention relates to the use of plants, seeds and propagation material derived from such plants as germplasm in a breeding program.

### BACKGROUND OF THE INVENTION

The eggplant, or aubergine, (Solanum melongena L.) is a plant of the nightshade family (Solanaceae) and belongs to the genus Solanum. It is closely related to tomato and potato. The eggplant bears a fruit of the same name, commonly used in cooking. Different varieties of the plant produce fruit of different sizes, shapes, and colors. The eggplant is an important food crop, grown worldwide in all conditions and climates both in protected cultivation and in the open field.

Eggplant fruit are popular for their pleasantly bitter taste and spongy texture. While the skin of the fruit is glossy and may vary in color from white, yellow, green through degrees of purple pigmentation to almost black, the flesh is white or cream colored and spongy in texture. Contained within the flesh of the fruit are seeds arranged in a conical pattern. Though eggplant seeds early in seed development have a white or cream color, at the commercially acceptable fruit stage (the stage at which eggplant fruit are harvested to be sold for human consumption) eggplant seeds have a brown color. At that stage the seeds in the eggplant fruit are not fully mature yet. The seeds reach the mature stage at which their brown color is most intense only when the eggplant fruit is at a stage that would be considered overripe by consumers. The brown color of eggplant seeds is due to the outermost layer of the seeds, the seed coat (also called testa), containing brown pigments.

The seed coat in many species contains dark (brown to black) pigments. Seed coat coloring has been studied best in Arabidopsis thaliana. In Arabidopsis seeds, pigmentation of the seed coat is observed at late stages of seed development. However, the actual synthesis of the pigments, which are called proanthocyanidins (PA) or condensed tannins, starts during early stages of embryo development (1-2 days after fertilisation). These flavonoids initially accumulate as colorless compounds in vacuoles of the endothelium, the innermost cell layer of integuments, are oxidized during seed desiccation, and confer the brown color to mature seeds. Several Arabidopsis seed coat pigmentation mutants are known. In these so called transparent testa mutants the Arabidopsis seed coat exhibits a white to pale yellow color. Many TRANSPARENT TESTA genes encode enzymes in the flavonoid biosynthesis pathway, while others encode regulatory genes involved in several points of the pathway. Most genes in the flavonoid pathway are single copy genes. In many transparent testa mutants the color of other parts of the plant (e.g. flowers and fruit) is affected, as besides having a disrupted proanthocyanidin accumulation in the seed coat their flavonol and/or anthocyanin production is disrupted in other plant parts or throughout the plant.

Citation or identification of any document in this application is not an admission that such document is available as prior art to the present invention.

### SUMMARY OF THE INVENTION

As the brown seeds of the eggplant fruit at the commercially acceptable fruit stage stand out among the white or creamy white flesh and make the eggplant fruit look less attractive to customers, having fruits produce seeds with a lighter color is a preferred trait. Especially if this lighter seed color is independent of the color of the skin of the fruit.

It is thus an object of the present invention to provide a new eggplant plant which produces seeds with a new color, in particular seeds with a lighter color.

In the research leading to the invention a new eggplant plant was developed that was found to produce seeds with a novel color, in particular a lighter color as further defined herein. It was found that production of seeds with this lighter color correlated with the homozygous presence in the Solanum melongena genome of a QTL, which QTL is as present in and obtainable from plants that are grown from seeds of which a representative sample was deposited with the NCIMB under accession number 42054. It was found that in the genome of plants grown from seeds of the deposit each of the markers of SEQ ID No:1, SEQ ID No:3, SEQ ID No:5, SEQ ID No:7, SEQ ID No:9, SEQ ID No:11, SEQ ID No:13 and SEQ ID No:15 (full sequence data given in Table 1) is linked to the QTL that causes the novel seed color of the invention. Although any of these markers or any combination of these markers may be used for identifying the QTL causing the invented trait of novel seed color, markers SEQ ID No:11 and SEQ ID No:13 are preferred because they have the highest LOD score in the statistical tests.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### DEPOSIT

Seeds of Solanum melongena plants with the QTL that leads to the eggplant plant producing seeds with a novel seed color were deposited at NCIMB Ltd., Ferguson Building, Craibstone Estate, Bucksburn, Aberdeen, AB21 9YA, UK, on Jan. 5, 2016 under NCIMB deposit number NCIMB 42507. Seeds of this deposit comprise the QTL homozygously. Plants grown from these seeds produce seeds with the novel seed color as defined herein.

The deposited seeds do not meet the DUS criteria which are required for obtaining plant variety protection, and can therefore not be considered to be plant varieties.

The Deposits with NCIMB Ltd., under deposit accession number NCIMB 42507 were made pursuant to the terms of the Budapest Treaty. Upon issuance of a patent, all restrictions upon the deposit will be removed, and the deposit is intended to meet the requirements of 37 CFR §§ 1.801-1.809. The deposit will be irrevocably and without restriction or condition released to the public upon the issuance of a patent and for the enforceable life of the patent. The deposit will be maintained in the depository for a period of 30 years, or 5 years after the last request, or for the effective life of the patent, whichever is longer, and will be replaced if necessary during that period.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1: Cross-section of an eggplant fruit of the invention at the commercially acceptable stage (B) and a wild-type eggplant fruit at the commercially acceptable stage (A) showing the seeds inside.

FIG. 2: Close up of a cross-section of an eggplant fruit of the invention at the commercially acceptable stage (B) and a wild-type eggplant fruit at the commercially acceptable stage (A) showing the seeds inside.

FIG. 3: Dried mature eggplant seeds of the invention (A) and dried mature wild-type eggplant seeds (B).

FIG. 4: Wild-type mature eggplant seed taken apart to show (A) the seed coat, (B) an intact half of the seed, and (C) the embryo plus endosperm of half of the seed. Mature eggplant seed of the invention taken apart to show (A) the seed coat, (B) an intact half of the seed, and (C) the embryo plus endosperm of half of the seed.

### DETAILED DESCRIPTION OF THE INVENTION

The presence of the QTL causing the invented trait of novel seed color in the genome of a plant can be determined by using the combination of markers of SEQ ID No:1, SEQ ID No:3, SEQ ID No:5, SEQ ID No:7, SEQ ID No:9, SEQ ID No:11, SEQ ID No:13 and SEQ ID No:15, the combination of markers of SEQ ID No:1, SEQ ID No:3, SEQ ID No:5, SEQ ID No:7, SEQ ID No:9, SEQ ID No:11 and SEQ ID No:13, the combination of markers of SEQ ID No:1, SEQ ID No:3, SEQ ID No:5 and SEQ ID No:7, the combination of markers of SEQ ID No:9, SEQ ID No:11, SEQ ID No:13 and SEQ ID No:15, the combination of markers of SEQ ID No:9, SEQ ID No:11 and SEQ ID No:13, or the combination of markers of SEQ ID No:11 and SEQ ID No:13.

The QTL is thus present in the genome of the deposited material and this material is thus a source of the QTL that can be used to introgress the novel seed color trait into other eggplant plants. Such plants may be used as a starting point to develop further varieties with the novel seed color. Any Solanum melongena plant comprising the QTL of the invention, regardless of the source of this QTL, is a plant of the invention.

“Introgression” as used herein is intended to mean introduction of a trait, by introgressing the QTL leading to that trait, into a plant not carrying the trait by means of crossing and selection in a generation in which the trait becomes visible, or in which the QTL can be selected. Such introgression is facilitated by the availability of markers, such as in-fragment markers.

In the deposited seeds, the QTL is linked with each of the molecular markers of SEQ ID No:1, SEQ ID No:3, SEQ ID No:5, SEQ ID No:7, SEQ ID No:9, SEQ ID No:11, SEQ ID No:13 and SEQ ID No:15. These markers may also be linked to the QTL that is comprised in either or both eggplant plants that are used as parents in a cross to transfer the novel seed color trait to other plants, but the presence of at least one of the mentioned markers is not essential as long as the QTL causing the trait is present. The presence of the novel seed color phenotype is a direct indicator that the QTL of the invention is present since the QTL is the genetic information that encodes the novel seed color trait. Thus, a plant of the invention which has the novel seed color trait as described herein is still a plant of the invention when the QTL underlying the phenotype is present therein but the markers no longer are. Markers are sometimes but not always the genetic cause of a trait. Markers may be located in the gene that causes the trait or are genetically linked to it. They are often used as tools to follow the inheritance of the trait. During breeding, the molecular markers that in the deposited seeds are linked to the genetic determinant may thus be used to assist transfer of the novel seed color trait to other plants. A skilled breeder would understand that the transfer of the novel seed color trait into an eggplant plant may be monitored by visual inspection, spectrophotometry or image analysis, or by monitoring and breeding for the presence of molecular markers as described herein (i.e. marker assisted selection), or both. Localization of such markers to specific genomic regions further allows for the use of associated sequences in breeding and for the development of additional linked genetic markers. Accordingly, it is emphasized that the present invention may be practiced using any molecular marker that genetically maps within the identified region provided that the marker is polymorphic between the parents of a cross to transfer the novel seed color trait to other plants.

The present invention thus provides an eggplant plant (Solanum melongena L.) comprising a QTL which when homozygously present leads to the eggplant plant producing seeds with the novel color as defined herein, wherein said QTL is the same as or similar to a QTL that is present in the genome of plants grown from seeds of deposit number NCIMB 42507 and is linked therein to at least one marker selected from the group consisting of SEQ ID No:1, SEQ ID No:3, SEQ ID No:5, SEQ ID No:7, SEQ ID No:9, SEQ ID No:11, SEQ ID No:13 and SEQ ID No:15, and wherein the QTL is preferably homozygously present. Said QTL is as present in the genome of, and obtainable from, eggplant plants grown from seeds of which a representative sample was deposited at the NCIMB under accession number NCIMB 42507.

In this context “eggplant” is intended to comprise Solanum melongena L.

The trait of the present invention, which trait is the reduction or complete absence of brown pigments in the seed coat of the seeds produced, leading to seeds having a lighter color, is a recessive trait. The QTL of the invention must thus be present in a homozygous state in the eggplant plant of the invention to result in the trait of the invention being visible in the seeds said plant produces.

The seed coat is the outer protective layer of the seed and is derived from the integuments of the ovule. The seed coat is thus of diploid maternal origin. The color of the seeds therefore is determined by the genotype of the plant that produces the seeds (the mother plant that receives pollen in a cross).

The invention also relates to eggplant plants of the invention, obtainable by crossing a first eggplant plant with a second eggplant plant, wherein at least one of the said plants comprises the QTL as comprised in an eggplant plant representative seed of which was deposited with the NCIMB under accession number NCIMB 42507, or a progeny plant thereof carrying the QTL, and selecting by visual inspection, preferably in the F2, for the trait of the invention, and/or by selecting for the presence of molecular markers as described herein.

The trait of the invention may be brought into a wild type eggplant plant by crossing the wild type plant, not carrying the QTL of the invention, with a plant that is either homozygous or heterozygous for the QTL of the invention and selecting for the desired phenotype, preferably in the F2 of that cross, and/or selecting for the presence of molecular markers as described herein, preferably in the F1 and/or F2 of that cross. The F2 is obtained by selfing the F1.

The eggplant plant into which the trait of the invention may be introduced may be an eggplant plant of any type, any fruit form or fruit color.

The novel seed color of seeds produced by plants carrying the QTL of the invention homozygously is defined as the seeds having a lighter color at the mature seed stage, as compared to the color of seeds produced by a plant not comprising the QTL homozygously. The seed coats of said seeds have a reduced amount of the brown pigments that are present in the seed coats of seeds at the mature seed stage produced by plants not comprising the QTL homozygously. In particular, the seed coats of said seeds have such a low amount of the brown pigments that are normally present in the seed coats of wild type seeds that the brown color in the seed coats of said seeds of plants of the invention is not detectable by the eye. More in particular, said seed coats completely lack the brown pigments that are normally present in the seed coats of wild type seeds.

Plants of the invention carrying the genetic determinant homozygously may thus be identified by the novel color of their seeds. The color difference between seeds produced by plants of the invention carrying the QTL of the invention homozygously and seeds of isogenic plants carrying the genetic determinant either heterozygously or not at all is easily observable by a skilled eggplant grower. When comparing the color of seeds produced by plants of the invention carrying the QTL of the invention homozygously and seeds of isogenic plants carrying the genetic determinant either heterozygously or not at all, all seeds have to be at the same developmental stage. The color difference of the seeds may be assessed at the commercially acceptable fruit stage or, preferably, at the mature seed stage. Though the color difference between seeds produced by plants of the invention carrying the QTL of the invention homozygously and seeds of isogenic plants carrying the genetic determinant either heterozygously or not at all is clear already at the commercially acceptable fruit stage, it is most clear at the mature seed stage.

The term “mature seed” is clear to the skilled eggplant grower and means in particular the seed stage of fully physiologically developed seeds. Mature seeds are at the seed stage as present in the fully physiologically developed eggplant fruit. Such a fully physiologically developed eggplant fruit is past the commercially acceptable fruit stage—the stage at which eggplant fruit are harvested to be sold for human consumption—and would be considered as overripe by consumers. The skilled eggplant grower knows when an eggplant fruit is at the commercially acceptable fruit stage and when an eggplant fruit is fully physiologically developed.

The color of the mature seeds of plants of the invention carrying the genetic determinant homozygously is much lighter than that of isogenic plants carrying the genetic determinant either heterozygously or not at all. This lighter color of mature seeds of plants carrying the genetic determinant homozygously, as compared to isogenic plants carrying the genetic determinant either heterozygously or not at all, is most apparent in fresh seeds immediately after harvest of the fruit.

The term “fresh seed” is also clear to the skilled person and means in particular the seed as present in the eggplant fruit immediately after harvest or taken from the fruit very recently and still wet. In other words, fresh seeds are those seeds that are not dried yet.

While the color of fresh mature wild type eggplant seeds not carrying the QTL of the invention may be called middle brown, sandy brown, bronze, or copper, the color of fresh mature seeds of plants of the invention carrying the genetic determinant homozygously may be indicated as off-white, creamy white, beige, very light yellow or light grayish yellow.

Upon drying of the seeds the color of both wild type seeds and seeds of plants of the invention changes. However, also when completely dry the color of seeds of plants of the invention is clearly distinguishable from and lighter than that of dried wild type seeds. While the color of dried mature wild type eggplant seeds not carrying the QTL of the invention may be called brown, sandy brown, middle brown, bronze or copper, the color of dried mature seeds of plants of the invention carrying the genetic determinant homozygously may be indicated as beige, light yellow, pale yellow, buff or khaki. Upon comparing the color of the seeds it is clear that this has to be done in the same stage, i.e. comparing fresh seeds to fresh seeds and dried seeds to dried seeds and also comparing seeds at the mature seed stage with seeds at the mature seed stage and seeds at the commercially acceptable fruit stage with seeds at the commercially acceptable fruit stage.

The novel seed color of the invention may thus be defined as fresh seeds produced by a plant of the invention having a lighter color at the mature seed stage, as compared to seeds produced by a plant not comprising the QTL homozygously and/or as dried seeds produced by said plants having a lighter color at the mature seed stage, as compared to seeds produced by a plant not comprising the QTL homozygously.

The novel seed color phenotype of seeds of the invention is due to the reduction or absence of brown pigments in the seed coat, also called testa, revealing the color of the cotyledons beneath the seed coat.

The coloration of the seeds of the invention and wild type seeds may also be determined using the RHS color chart (The Royal Horticultural Society, London, UK). The color of fresh mature wild type seeds not carrying the QTL of the invention resembles the color 165B at its darkest parts and 165C at its lightest parts, while the color of fresh mature seeds of the invention resembles the color 161D at its lightest parts and 161C at its darkest parts (Table 2). The color of dried mature wild type seeds resembles the color 162A, while the color of dried mature seeds of the invention resembles the color 164B (Table 2).

Though the RHS color chart is commonly used by plant breeders and growers for determining plant colors, it is clear the color may also be determined using other color charts or systems. Colors may, for example, also be specified in RGB color codes, using the Munsell color system or may be determined using a colorimeter or image analysis. The skilled person knows how to use these different color systems and convert color codes between different color systems. The RGB, CIELab and CIELCh values for these RHS colors are listed in the table below.

| | | | | CIE Lab | | | CIE LCh | | |
|---|---|---|---|---|---|---|---|---|---|
| | sRGB | | | D65/10° | | | D65/10° | | |
| RHS | R | G | B | L | a | b | L | C | h |
| 161C | 228 | 195 | 145 | 80 | 5 | 29 | 80 | 29 | 80 |
| 161D | 233 | 205 | 163 | 84 | 4 | 24 | 84 | 24 | 81 |
| 162A | 223 | 172 | 91 | 74 | 10 | 47 | 74 | 48 | 78 |
| 164B | 201 | 142 | 84 | 64 | 16 | 39 | 64 | 42 | 67 |
| 165B | 174 | 111 | 62 | 53 | 20 | 37 | 53 | 42 | 61 |
| 165C | 208 | 150 | 99 | 67 | 16 | 35 | 67 | 38 | 65 |

A color scale that is widely used to measure colors, for instance using a colorimeter or image analysis, is the CIELAB color scale. The scale includes 3 data variables: L*, a* and b*. L* indicates lightness on a 0 to 100 scale, where 0 is black and 100 is white. The variables a* and b* indicate the amount of red, green, blue and yellow color: a* value indicates color change from green (negative values) to red (positive values), while b* indicates color change from yellow (positive values) to blue (negative values). Differences in color between two samples can be expressed in terms of change in L* and/or a*, and/or b*.

As is clear to the skilled person color parameters vary depending on the method used for quantifying colors. Still, when comparing the L* (10°/D65) scores for a particular color quantification method (either color estimation using an RHS color chart, colorimetry or image analysis) of wild type fresh mature seed with fresh mature seed of the invention, the L* score is consistently higher for the seed of the invention, when compared to the wild type seed not carrying the QTL of the invention. Also when comparing the L* (10°/D65) scores for a particular color quantification method (either color estimation using an RHS color chart, colorimetry or image analysis) of wild type dried mature seed with dried mature seed of the invention the L* score is always higher for the seed of the invention, when compared to the wild type seed not carrying the QTL of the invention. A higher L* score indicates a lighter color.

In one embodiment the invention relates to an eggplant (Solanum melongena) plant homozygously comprising the QTL of the invention, wherein the L* (10°/D65) score when determined using an RHS color chart and converting the RHS color code to the corresponding CIELAB color scale on dry mature seeds of said plant is at least, in order of increased preference, 47, 50, 54, 58, 59, 65, 70, 80, 90. The L* (10′/D65) score when determined using an RHS color chart on dry mature seeds of said plant is suitably not higher than 99.

In one embodiment the invention relates to an eggplant (Solanum melongena) plant homozygously comprising the QTL of the invention, wherein the L* (10′/D65) score when determined using an RHS color chart and converting the RHS color code to the corresponding CIELAB color scale on fresh mature seeds of said plant is at least, in order of increased preference, 49, 52, 57, 63, 67, 70, 80, 90. The L* (10°/D65) score when determined using an RHS color chart on fresh mature seeds of said plant is suitably not higher than 99.

In one embodiment the invention relates to an eggplant (Solanum melongena) plant homozygously comprising the QTL of the invention, wherein the L* (10°/D65) score when determined using colorimeter on dry mature seeds of said plant is at least, in order of increased preference, 61.5, 62, 63, 65, 70, 80, 90. The L* (10°/D65) score when determined using colorimeter on dry mature seeds of said plant is suitably not higher than 99.

In one embodiment the invention relates to an eggplant (Solanum melongena) plant homozygously comprising the QTL of the invention, wherein the L* (10°/D65) score when determined using colorimeter on fresh mature seeds of said plant is at least, in order of increased preference, 85, 87, 89, 90. The L* (10°/D65) score when determined using colorimeter on fresh mature seeds of said plant is suitably not higher than 99.

In one embodiment the invention relates to an eggplant (Solanum melongena) plant homozygously comprising the QTL of the invention, wherein the L* (10°/D65) score when determined using image analysis, e.g. as described in example 3, on dry mature seeds of said plant is at least, in order of increased preference, 55, 58, 60, 62, 65, 70, 80, 90. The L* (10°/D65) score when determined using colorimeter on dry mature seeds of said plant is suitably not higher than 99.

In one embodiment the invention relates to an eggplant (Solanum melongena) plant homozygously comprising the QTL of the invention, wherein the L* (10°/D65) score when determined using image analysis, e.g. as described in example 3, on fresh mature seeds of said plant is at least, in order of increased preference, 62, 64, 66, 68, 70, 80, 90. The L* (10°/D65) score when determined using colorimeter on fresh mature seeds of said plant is suitably not higher than 99.

Thus, as used herein the term “novel seed color” is intended to refer to a seed color of fresh mature seeds that is off-white, creamy white, beige, very light yellow or light grayish yellow and/or showing color 161D according to the RHS color code at its lightest parts and 161C according to the RHS color code at its darkest parts, and a seed color of dried mature seeds that is beige, light yellow, pale yellow, buff or khaki and/or showing the color 164B according to the RHS color code.

The invention relates also to seed of the eggplant plant of the invention and to other parts of the plant that are suitable for sexual reproduction. Such a plant part may be selected from the group consisting of a microspore, pollen, an ovary, an ovule, an embryo sac and an egg cell.

Additionally, the invention also relates to parts of the eggplant plant of the invention that are suitable for vegetative reproduction, for example a tissue culture, a cutting, a root, a stem, a cell and a protoplast. Tissue culture may be grown from a leaf, pollen, an embryo, a cotyledon, a hypocotyl, a meristematic cell, a root, an anther, a flower, a seed or a stem.

The invention further relates to a seed of an eggplant plant of the invention comprising a QTL which when homozygously present leads to the eggplant plant producing seeds with a novel color. In such a seed the QTL may be homozygously or heterozygously present, but is preferably homozygously present.

The invention also relates to a seed that is capable of growing into an eggplant plant of the invention, wherein the genome of the pepper seed comprises a QTL, that when homozygously present in the plant that can be grown from the seed causes the trait of the novel seed color. In such a seed the QTL may be homozygously or heterozygously present, but is preferably homozygously present.

The invention furthermore relates to hybrid seed and to a method of producing hybrid seeds comprising crossing a first parent plant with a second parent plant and harvesting the resulting hybrid seed. Such hybrid seed may either comprise the QTL of the invention heterozygously or homozygously. In order for all the hybrid seeds to carry the trait of the invention homozygously so that plants grown from said hybrid seeds produce seeds with a novel color, both parents need to be homozygous for the QTL causing the trait of novel seed color. Both parents thus carry the QTL of the invention. They need not necessarily be uniform for other traits.

Besides the seed of an eggplant plant, the invention also covers the progeny derived from an eggplant plant comprising a QTL which when present homozygously leads to the eggplant producing seeds with a novel color. Such progeny may be produced by sexual or vegetative reproduction of a plant of the invention or a progeny plant thereof. The progeny carries the QTL as found in the plant of the invention and as present in a plant grown from a seed of which a representative sample was deposited at the NCIMB under number NCIMB 42507. The said QTL may be present in the progeny both homozygously and heterozygously, preferably the first option. In addition to this, the plant may be modified in one or more characteristics other than the seed color. Such additional modifications are for example effected by crossing and selecting, mutagenesis or by transformation with a transgene.

As used herein the word “progeny” is intended to mean the offspring or the first and all further descendants from a cross of any eggplant plant with a plant of the invention that carries the QTL of the invention underlying the novel seed color trait. Progeny of the invention comprises descendants of any cross with a plant of the invention that carries the QTL causing the novel seed color trait of the invention. Such progeny is for example obtainable by crossing a first eggplant plant with a second eggplant plant, wherein one of the plants was grown from a seed of which a representative sample was deposited under accession number NCIMB 42507, but may also be the progeny of any other eggplant plant carrying the QTL of the invention as present in NCIMB 42507.

Furthermore, the current invention also covers progeny of an eggplant plant of the current invention or progeny of an eggplant plant grown a from a seed derived from a plant of the current invention, wherein the progeny of the plant comprises the QTL and wherein the QTL is preferably present in a homozygous state, although the QTL may be present in heterozygous state.

Propagation material derived from an eggplant plant of the invention or from an eggplant seed from an eggplant plant of the invention, is also included in the present invention, wherein the propagation material comprises the QTL that when homozygously present causes the novel seed color, and the QTL is preferably present in a homozygous state.

The invention also refers to propagation material capable of growing into an eggplant plant of the invention, wherein the propagation material comprises the QTL that when homozygously present causes the novel seed color, and the QTL is preferably present in a homozygous state.

The said propagation material, derived from the eggplant plant of the invention as well as propagation material capable of growing into a plant of the invention is for example selected from the group consisting of callus, a microspore, pollen, an ovary, an ovule, an embryo, an embryo sac, an egg cell, a cutting, a root, a stem, a cell, a protoplast, a leaf, a cotyledon, a hypocotyl, a meristematic cell, a root, a root tip, a microspore, an anther, a flower, a seed and a stem, or a part or tissue culture thereof.

The invention further relates to a cell of an eggplant plant of the invention, which cell comprises the QTL which in a homozygous state confers the novel seed color trait, wherein said QTL is as present in the genome of an eggplant plant grown from seeds of which a representative sample was deposited with the NCIMB under accession number NCIMB 42507. Said eggplant plant is obtainable by crossing an eggplant plant with a second eggplant plant, in particular an eggplant plant grown from seed as deposited under accession number NCIMB 42507, and selecting, preferably in the F2, for an eggplant plant that has the novel seed color trait of the invention. The said cell thus comprises the genetic information which is substantially identical, preferably completely identical to the genetic information encoding the said novel seed color trait of an eggplant plant grown from a seed of which a representative sample was deposited under NCIMB accession number 42507, more in particular the QTL described herein. Preferably, the cell of the invention is a part of a plant or plant part, but the cell may also be in isolated form.

In one embodiment, the invention relates to the use of seeds with NCIMB accession number NCIMB 42507, for transferring the QTL of the invention, which confers the novel seed color trait of the invention, into another eggplant plant. The invention also relates to the use of eggplant plants that have the same QTL as the deposited seeds for transferring the QTL of the invention into another eggplant plant.

In another embodiment, the invention relates to the use of an eggplant plant, which plant carries the QTL of the invention as a crop.

The invention also relates to the use of an eggplant plant, which carries the QTL of the invention, as a source of seed.

In yet another embodiment, the invention relates to the use of an eggplant plant, which carries the QTL of the invention, as a source of propagating material.

Further, the invention relates to the use of an eggplant plant, which carries the QTL of the invention, for consumption.

In another embodiment, the invention relates to the use of an eggplant plant, which carries the QTL of the invention, for conferring the QTL to an eggplant plant.

In yet another embodiment, the invention relates to the use of an eggplant plant, as a recipient of the QTL of the invention, in particular an eggplant plant grown from seed with NCIMB accession number NCIMB 42507.

The current invention also relates to an eggplant fruit, or a part thereof, harvested from an eggplant plant of the invention, producing fruits with seeds with a novel seed color and comprising the QTL as defined herein. Naturally this also relates to any food product or processed food product made of said eggplant fruit.

The current invention also relates to an eggplant fruit, or parts thereof, harvested from an eggplant plant of the invention, comprising the QTL as defined herein in heterozygous state. Naturally this also relates to any food product or processed food product made of said eggplant fruit. Although the trait of novel seed color is not visible in such plants, they are still a source of the QTL and can for example be used in breeding.

The processed eggplant fruit may also be included in another food product, such as sauce, baba ghanoush, curry, pie, moussaka, soup or a dried or fresh pasta product, such as ravioli, tortellini, cannelloni etc. Such food product will usually be pre-packed and is intended for sale in a supermarket. The invention thus also relates to the use of eggplant fruits harvested from an eggplant plant of the invention, or parts thereof, in the preparation of food products, in particular sauces, salads, pies, soups and pastas. Also in processed form the eggplant fruit still comprises the QTL of the invention.

An eggplant plant of the invention could also be used as germplasm in a breeding program for the development of other eggplant plants that comprise the QTL that causes the novel seed color. This kind of use is also covered by the current invention.

Moreover, the invention relates to a nucleic acid or a part thereof, optionally in isolated form, which when present in a homozygous state causes the novel seed color in eggplant plants as defined herein, which nucleic acid originates from Linkage Group 11 based on the genome sequence given in Hirakawa et al., Draft Genome Sequence of Eggplant (Solanum melongena L.): the Representative Solanum Species Indigenous to the Old World, 2014, DNA Research 21, 649-660 and is linked thereon to at least one molecular marker selected from the group consisting of SEQ ID No:1, SEQ ID No:3, SEQ ID No:5, SEQ ID No:7, SEQ ID No:9, SEQ ID No:11, SEQ ID No:13 and SEQ ID No:15 and/or is present in seeds with NCIMB accession number NCIMB 42507. In particular said nucleic acid is linked to the combination of markers of SEQ ID No:1, SEQ ID No:3, SEQ ID No:5, SEQ ID No:7, SEQ ID No:9, SEQ ID No:11 and SEQ ID No:13, more in particular the combination of markers of SEQ ID No:9, SEQ ID No:11 and SEQ ID No:13, most in particular the combination of markers of SEQ ID No:11 and SEQ ID No:13 or the combination of markers of SEQ ID No:1, SEQ ID No:3, SEQ ID No:5 and SEQ ID No:7. A person skilled in the art would be able to isolate the nucleic acid causing the seed color trait of the invention or a part thereof from the genome of an eggplant plant of the invention, and use it to create new molecular markers that are linked with the QTL and with the trait of the invention.

The present invention also relates to the use of a molecular marker, wherein the marker is selected from the group consisting of SEQ ID No:1, SEQ ID No:3, SEQ ID No:5, SEQ ID No:7, SEQ ID No:9, SEQ ID No:11, SEQ ID No:13 and SEQ ID No:15 to identify the QTL in an eggplant plant of the invention, or to develop an eggplant plant with novel seed color.

The present invention further relates to the use of said molecular marker to identify or develop other markers linked to the QTL that causes the novel seed color.

In order to establish the presence of the QTL of the invention in the genome of a seed or plant at least one molecular marker is necessary but any combination of the molecular markers according to SEQ ID No:1, SEQ ID No:3, SEQ ID No:5, SEQ ID No:7, SEQ ID No:9, SEQ ID No:11, SEQ ID No:13 and SEQ ID No:15 may be used. Genotyping a population or collection of plants that is not uniform for the seed color trait of the invention may be done using at least one molecular marker set selected from the group consisting of SEQ ID No:1 plus SEQ ID No:2, SEQ ID No:3 plus SEQ ID No:4, SEQ ID No:5 plus SEQ ID No:6, SEQ ID No:7 plus SEQ ID No:8, SEQ ID No:9 plus SEQ ID No:10, SEQ ID No:11 plus SEQ ID No:12, SEQ ID No:13 plus SEQ ID No:14 and SEQ ID No:15 plus SEQ ID No:16.

The homozygous presence of the novel seed color QTL of the invention in a plant may also be determined phenotypically, by checking the color of the mature seeds produced by that plant. The presence of the novel seed color trait of the invention may be determined phenotypically on any plant or plant population. In an introgression process, determining the presence of the novel seed color trait of the invention phenotypically is most informative in any generation in which the trait is segregating, preferably in the F2, of an introgression process.

In one aspect the invention relates to a method for the production of an eggplant plant which has the trait of producing seeds with a novel color, comprising

a) crossing a plant, comprising a QTL that when homozygously present leads to the novel seed color, with another plant;

b) selecting plants that have the novel seed color in the F1 and/or F2 generation;

c) optionally performing one or more additional rounds of selfing and/or crossing and/or backcrossing with the preferred parent, and subsequently selecting, for a plant comprising/showing the trait of the invention.

Selecting plants that have the trait of producing seeds with a novel color can be done molecularly using molecular markers linked to the trait as described herein in the F1 or any further generation and/or phenotypically in the F2 or any further generation in which the trait segregates.

The word “trait” in the context of this application refers to the phenotype of the plant. In particular, the word “trait” refers to the trait of the invention, more in particular to the trait of eggplant plants producing seeds with a novel color. The term “QTL” (i.e. “quantitative trait locus”) is used for the genetic information in the genome of the plant that in a homozygous state causes the novel seed color trait of the invention. When the genome of a plant comprises the QTL causing the trait of the invention seed produced by the plant has the novel seed color of the invention. The plant thus has the QTL of the invention.

It is clear that the parent that provides the trait of the invention is not necessarily a plant grown directly from the deposited seeds. The parent may also be a progeny plant from the deposited seed, obtained by for example selfing or crossing, or a progeny plant from seeds that are identified to have the trait of the invention by other means.

The invention additionally provides a method of introducing another desired trait into an eggplant plant which has the trait of producing seeds with a novel color, comprising:

a) crossing an eggplant plant that has the trait of novel seed color, representative seeds of which were deposited under deposit number NCIMB 42507, with a second eggplant plant that comprises a desired trait to produce F1 progeny;

b) selecting in the F2 progeny plants that comprise said trait of novel seed color and the desired trait;

c) crossing the selected F2 progeny plants with either parent, to produce backcross progeny;

d) selecting backcross progeny plants comprising the desired trait and the trait of novel seed color; and

e) optionally repeating steps c) and d) one or more times in succession to produce selected fourth or higher backcross progeny that comprises the desired trait and the trait of novel seed color. The invention includes an eggplant plant produced by this method.

In one embodiment selection for plants having the trait of novel seed color is done in the F1 or any further generation by using any, or any combination, of the markers according to SEQ ID No:1, SEQ ID No:3, SEQ ID No:5, SEQ ID No:7, SEQ ID No:9, SEQ ID No:11, SEQ ID No:13 and SEQ ID No:15. In another aspect, selection for the trait of the invention is started in the F2 of a cross or alternatively of a backcross. Selection of plants in the F2 can be done phenotypically as well as by using the said marker(s) which directly or indirectly detect(s) the QTL underlying the trait. Phenotypic selection can suitably be done by determining the color of the seeds using an RHS color chart for reference and/or by determining the color profile by colorimetry and/or image analysis of the eggplant seeds.

In one embodiment selection for plants having the trait of novel seed color is started in the F3 or a later generation.

In one embodiment the plant comprising the QTL is a plant of an inbred line, a hybrid, a doubled haploid, or of a segregating population.

In one embodiment the plant of the invention, i.e. a plant which comprises the QTL of the invention, is an agronomically elite eggplant plant.

In the context of this invention an agronomically elite eggplant plant is a plant having a genotype that as a result of directed crossing and selection by human intervention results into an accumulation of distinguishable and desirable agronomic traits which allow a producer to harvest a product of commercial significance.

In the course of breeding a new eggplant plant carrying the QTL of the invention, desirable agronomic traits may be introduced into said eggplant plant independently of the QTL of the invention. As used herein, “desirable traits” include but are not limited to e.g. improved yield, fruit shape, fruit size, fruit color, seed size, plant vigor, plant height, and resistance to one or more diseases or disease causing organisms. Any one of these desirable traits may be combined with the QTL of the invention.

In yet a further embodiment, the agronomically elite eggplant plant of the invention is an inbred line or a hybrid.

As used herein, a plant of an inbred line is a plant of a population of plants that is the result of three or more rounds of selfing, or backcrossing; or which plant is a double haploid. An inbred line may e.g. be a parent line used for the production of a commercial hybrid.

As used herein, a hybrid plant is a plant which is the result of a cross between two different plants having different genotypes. More in particular, a hybrid plant is the result of a cross between plants of two different inbred lines, such a hybrid plant may e.g. be a plant of a commercial F1 hybrid variety.

In one embodiment the plant which comprises the QTL of the invention is an F1 hybrid variety.

The invention further provides a method for the production of an eggplant plant having the trait of novel seed color by using a doubled haploid generation technique to generate a doubled haploid line comprising said trait.

The invention furthermore relates to hybrid seed that can be grown into a plant having the trait of novel seed color and to a method for producing such hybrid seed comprising crossing a first parent plant with a second parent plant and harvesting the resultant hybrid seed, wherein said first parent plant and/or said second parent plant is the plant as claimed.

In one embodiment, the invention relates to a method for producing a hybrid eggplant plant that has the trait of novel seed color, comprising crossing a first parent eggplant plant with a second parent eggplant plant and harvesting the resultant hybrid seed, of which the first parent plant and the second parent plant has the trait of novel seed color, and growing said hybrid seeds into hybrid plants having the trait of novel seed color.

The invention also relates to a method for the production of an eggplant plant having the trait of novel seed color by using a seed that homozygously comprises the QTL of the invention for growing the said eggplant plant. The seeds are suitably seeds of which a representative sample was deposited with the NCIMB under deposit number NCIMB 42507.

The invention also relates to a method for seed production comprising growing eggplant plants from seeds of which a representative sample was deposited with the NCIMB under deposit number NCIMB 42507, allowing the plants to produce seeds, and harvesting those seeds. Production of the seeds is suitably done by crossing or selfing.

In one embodiment, the invention relates to a method for the production of an eggplant plant having the trait of producing seeds with a novel color by using tissue culture.

The invention furthermore relates to a method for the production of an eggplant plant having the trait of producing seeds with a novel color by using vegetative reproduction.

In one embodiment, the invention relates to a method for the production of an eggplant plant having the trait of producing seeds with a novel color by using a method for genetic modification to introgress the QTL causing the said trait into the eggplant plant. Genetic modification comprises transgenic modification or transgenesis, using a gene from a non-crossable species or a synthetic gene, and cisgenic modification or cisgenesis, using a natural gene, coding for an (agricultural) trait, from the crop plant itself or from a sexually compatible donor plant.

In one embodiment, the source from which the genetic information is acquired, in particular the QTL, is formed by a plant grown from the deposited seeds, or by sexual or vegetative descendants thereof.

The invention also relates to a breeding method for the development of eggplant plants that have the trait of producing seeds of a novel color wherein germplasm comprising the QTL causing said trait is used. The germplasm is constituted by all inherited characteristics of an organism and according to the invention encompasses at least the novel seed color trait of the invention. Representative seed of said plant comprising the QTL and being representative for the germplasm was deposited with the NCIMB under deposit number NCIMB 42507.

In a further embodiment the invention relates to a method for the production of an eggplant plant having the trait of producing seeds with a novel color wherein progeny or propagation material of a plant comprising the QTL conferring said trait is used as a source to introgress the said trait into another eggplant plant. Representative seed of said plant comprising the QTL was deposited with the NCIMB under deposit number NCIMB 42507.

Furthermore, the invention relates to a novel seed color gene that leads to an eggplant plant having the novel seed color trait of the invention, which novel seed color gene is as present in the genome of plants of which a representative sample was deposited with the NCIMB under deposit number NCIMB 42507. The skilled breeder knows how to use such a plant as a source of the novel seed color gene for introgressing the novel seed color gene into a plant.

The invention also relates to the use of the QTL that leads to an eggplant plant having the trait of producing seeds with a novel seed color, for producing a plant which has the trait of novel seed color, in particular an eggplant plant which has the novel seed color trait, which QTL is as present in the genome of plants of which a representative sample was deposited under deposit number NCIMB 42507.

According to another aspect thereof the invention relates to a non-naturally occurring plant producing seeds having a novel seed color, which novel seed color is the result of the presence in the genome of the plant of the QTL which is as present in the genome of plants of which a representative sample was deposited under deposit accession number 42507. The non-naturally occurring plant is in particular a mutant plant.

The term ‘nucleic acid’ is used for a macromolecule, a DNA or RNA molecule, containing the genetic information that causes the trait of the invention. When a plant shows the phenotypic trait of the invention, its genome comprises the nucleic acid causing that trait. The plant thus has the nucleic acid of the invention. In the present invention the nucleic acid is part of the QTL which is as present in the genome of plants of which a representative sample was deposited under deposit number NCIMB 42507.

The invention provides preferably an eggplant plant having the trait of novel seed color, which plant is obtainable by any of the methods described herein and/or familiar to the skilled person.

In the absence of molecular markers, or in the instance that recombination between the QTL and the marker has taken place so that the marker is not predictive anymore for the presence of the QTL in the genome of the plant, equivalence of a genetic determinant with the QTL of the invention can be determined by an allelism test. To perform an allelism test, material that is homozygous for the known QTL of the invention, i.e. a tester plant, is crossed with material that is homozygous for the genetic determinant to be tested. This latter plant is referred to as the donor plant. The donor plant to be tested should be or should be made homozygous for the determinant to be tested. The skilled person knows how to obtain a plant that is homozygous for the determinant to be tested. When in the F2 of the cross between a donor plant and a tester plant no segregation for the phenotype related to the locus of the invention is observed, the determinant of the donor plant and the known locus of the tester plant have been proven to be equivalent. The phenotype that should be observed is a novel seed color, in particular a lighter seed color as defined herein. A tester plant may be, but is not limited to, a plant grown from seed deposited with the NCIMB under accession number 42054.

When more than one gene may be responsible for a certain trait, and an allelism test is done to determine equivalence, the skilled person doing the test has to make sure that all relevant genes are present homozygously for the test to work properly.

TABLE 1
Molecular SNP markers

| | | posi- | |
|---|---|---|---|
| marker | L | tion | |
| name | G | (cM) | Sequence marker |
| SEQ ID | 11 | 34.4 | TTTTTTAGACATTTGCATCAACTTTGCAGACG |
| No: 1 | | | ATACTTAGGAATCACATAAGATGTTGAACTCT |
| Linked | | | AGCTGTTACTAAACCGAATTTCTCAGTCATGT |
| to the | | | CAATATCCTTCGGTGTCATGCCATTTGGCAAC |
| QTL | | | TCCCAGTCAAAGCAATGA |
| SEQ ID | 11 | 34.4 | TTTTTTAGACATTTGCATCAACTTTGCAGACG |
| No: 2 | | | ATACTTAGGAATCAACATAAGATGTTGAACTC |
| Wild- | | | TAGCTGTTACTAAACCGAATTTCTCAGTCATG |
| type | | | TCAATATCCTTCGGTGTCATGCCATTTGGCAA |
| | | | CTCCCAGTCAAAGCAATGA |
| SEQ ID | 11 | 34.4 | CCTTCGGTGTCATGCCATTTGGCAACTCCCAG |
| No: 3 | | | TCAAAGCAATGAACCAATTGTGCGAGCACCA |
| Linked | | | AACGAACCGTGGTGAGCCCCAACTGCAAACC |
| to the | | | CGGGCAGCTTCTTCTGCCCGAACCAAATGGTA |
| QTL | | | AAAGCTCAAAATGTCGCCCGTGAAGGTCAAT |
| | | | GTTGCTTCCTTGAAATCTCT |
| SEQ ID | 11 | 34.4 | CCTTCGGTGTCATGCCATTTGGCAACTCCCAG |
| No: 4 | | | TCAAAGCAATGAACCAATTGTGCGAGCACCA |
| Wild- | | | AACGAACCGTGGTGAGCCCCAACTGCAAACC |
| type | | | CGGGCAACTTCTTCTGCCCGAACCAAATGGTA |
| | | | AAAGCTCAAAATGTCGCCCGTGAAGGTCAAT |
| | | | GTTGCTTCCTTGAAATCTCT |
| SEQ ID | 11 | 34.4 | CTTCTTCTGCCCGAACCAAATGGTAAAAGCTC |
| No: 5 | | | AAAATGTCGCCCGTGAAGGTCAATGTTGCTTC |
| Linked | | | CTTGAAATCTCTTAGGTTTGAACTTTTCTGGC |
| to the | | | TCAACCCAAATGTCTGGATCTCTTCCTATTGT |
| QTL | | | CCAAACATTGATGAGAAGTCGGGATCCTTTG |
| | | | GGTATGTGAAAACCATCA |
| SEQ ID | 11 | 34.4 | CTTCTTCTGCCCGAACCAAATGGTAAAAGCTC |
| No: 6 | | | AAAATGTCGCCCGTGAAGGTCAATGTTGCTTC |
| Wild- | | | CTTGAAATCTCTCAGGTTTGAACTTTTCTGGC |
| type | | | TCAACCCAAATGTCTGGATCTCTTCCTATTGT |
| | | | CCAAACATTGATGAGAAGTCGGGATCCTTTG |
| | | | GGTATGTGAAAACCATCA |
| SEQ ID | 11 | 34.4 | CGACTCGTGGGGAATCAACAAGGGTGCAACA |
| No: 7 | | | GGATGGAGTCTACTACTTTCTTTTATGACCAT |
| Linked | | | ATCTAAATATTCAAGATTTTCTAAGTCTGATT |
| to the | | | CTTCTACCATTCTGTTTTTGCCAGCTACTTGTT |
| QTL | | | CCAACTCTTTTTGAAGCCTTTTCATCACATTG |
| | | | GGGTGCCTTAGAAGTTCTGCTAAAATCCATTC |
| | | | TATTGTTGT |
| SEQ ID | 11 | 34.4 | CGACTCGTGGGGAATCAACAAGGGTGCAACA |
| No: 8 | | | GGATGGAGTCTACTACTTTCTTTTATGACCAT |
| Wild- | | | ATCTAAATATTCAAGATTTTCTAAGTCTGATT |
| type | | | CTTCTGCCATTCTGTTTTTGCCAGCTACTTGTT |
| | | | CCAACTCTTTTTGAAGCCTTTTCATCACATTG |
| | | | GGGTGCCTTAGAAGTTCTGCTAAAATCCATTC |
| | | | TATTGTTGT |
| SEQ ID | 11 | 34.4 | TAAACTAGGGAGGTTCTGCCGGCCTGCAATTT |
| No: 9 | | | TCAAGTTCCATGGCTCCTCAGAACCACCACCA |
| Linked | | | ACTTCCTTGAGTTTCTCTAATGATCACTTGCTC |
| to the | | | ACATCGAACTCGAGATCTCTGTCTGGGCAAAT |
| QTL | | | TGAGTCTGGTACAGAAATTGTTGAGCATGTTG |
| | | | TTCCAGAGGGAGTTACTGCAGTTTCAGCATCT |
| | | | ACACATCT |
| SEQ ID | 11 | 34.4 | TAAACTAGGGAGGTTCTGCCGGCCTGCAATTT |
| No: 10 | | | TCAAGTTCCATGGCTCCTCAGAACCACCACCA |
| Wild- | | | ACTTCCTTGAGTTTCTCTAATGATCACTTGCTC |
| type | | | ACAGCGAACTCGAGATCTCTGTCTGGGCAAA |
| | | | TTGAGTCTGGTACAGAAATTGTTGAGCATGTT |
| | | | GTTCCAGAGGGAGTTACTGCAGTTTCAGCATC |
| | | | TACACATCT |
| SEQ ID | 11 | 36.7 | CTCCGGAAACAACCTCTTTATCTCTGTGAGGT |
| No: 11 | | | AGTGATAAGGTCTGCGTACATTTCACCCTCCC |
| Linked | | | CAGATTCCACTGGGTATGTTGTTGTTGTTCAT |
| to the | | | GTATTATACTATAAGCTTCAATAAATCGAAAA |
| QTL | | | TTTGTTCATTACATTCCTTTTCTTGTTTATTTTT |
| | | | GACAGACAAGGAGAAGCATCTTCAGCTACTT |
| | | | CAACTGCT |
| SEQ ID | 11 | 36.7 | CTCCGGAAACAACCTCTTTATCTCTGTGAGGT |
| No: 12 | | | AGTGATAAGGTCTGCGTACATTTCACCCTCCC |
| Wild- | | | CAGATTCCACTGGGTATGTTGTTGTTGTTCAT |
| type | | | GTATCATACTATAAGCTTCAATAAATCGAAA |
| | | | ATTTGTTCATTACATTCCTTTTCTTGTTTATTTT |
| | | | TGACAGACAAGGAGAAGCATCTTCAGCTACT |
| | | | TCAACTGCT |
| SEQ ID | 11 | 36.7 | CGAACTGTGACATCAAGCACTGTTTGTAATGC |
| No: 13 | | | TTCAATCTGCAAACTGGAATCGTGACAAGCA |
| Linked | | | GACAAGCTAGGCTTGTTTTATAAGCTCAGCCA |
| to the | | | AATTACAGAGAGTAGTTGAAATATCTGAAAC |
| QTL | | | TCAAGCAGCCATGGGGTTCGGTTCTTGATCTT |
| | | | TTTGTACCAGAGTTCAACGACAAATACAAACT |
| | | | TTTGCTCACTA |
| SEQ ID | 11 | 36.7 | CGAACTGTGACATCAAGCACTGTTTGTAATGC |
| No: 14 | | | TTCAATCTGCAAACTGGAATCGTGACAAGCA |
| Wild- | | | GACAAGCTAGGCTTGTTTTATAAGCTCAGCCA |
| type | | | AATTAGAGAGAGTAGTTGAAATATCTGAAAC |
| | | | TCAAGCAGCCATGGGGTTCGGTTCTTGATCTT |
| | | | TTTGTACCAGAGTTCAACGACAAATACAAACT |
| | | | TTTGCTCACTA |
| SEQ ID | 11 | 40.8 | AACCAACACCAAAACCACCTGTTCATAGCAA |
| No: 15 | | | ACCACCACCACCTGGTTGGAAGTCTTCGGGTT |
| Linked | | | CGCATAATAAGCGATCTCCACCGCCACCTATA |
| to the | | | TCGACTCCTCTACCTTCACCACCACCACCAAC |
| QTL | | | TTACAAAATCTCTCCTACGCCTAGCCCTCCAT |
| | | | CTTCTTCTTCTTCTCCCCCACCACCTGTTTACA |
| SEQ ID | 11 | 40.8 | AACCAACACCAAAACCACCTGTTCATAGCAA |
| No: 16 | | | ACCACCACCACCTGGTTGGAAGTCTTCGGGTT |
| Wild- | | | CGCATAATAAGCGATCTCCACCGCCACCCAT |
| type | | | ATCGACTCCTCTACCTTCACCACCACCACCAA |
| | | | CTTACAAAATCTCTCCTACGCCTAGCCCTCCA |
| | | | TCTTCTTCTTCTTCTCCCCCACCACCTGTTTAC |
| | | | A |

The SNP sequences of the markers SEQ ID No:1, SEQ ID No:3, SEQ ID No:5, SEQ ID No:7, SEQ ID No:9, SEQ ID No:11, SEQ ID No:13 and SEQ ID No:15 are in the genome of seeds of the deposit NCIMB 42507 linked to the QTL of the invention, which confers the trait of a producing seeds with a novel seed color to eggplant plants. The sequences of SEQ ID No:2, SEQ ID No:4, SEQ ID No:6, SEQ ID No:8, SEQ ID No:10, SEQ ID No:12, SEQ ID No:14 and SEQ ID No:16 represent the wild type S. melongena alleles for the molecular SNP markers SEQ ID No:1, SEQ ID No:3, SEQ ID No:5, SEQ ID No:7, SEQ ID No:9, SEQ ID No:11, SEQ ID No:13 and SEQ ID No:15, respectively.

The nucleotides that differ between the marker linked to the trait of the invention and the wild type are underlined and in bold

The marker allele SEQ ID No:1 differs from marker allele SEQ ID No:2 not by a nucleotide difference, but by a single base pair deletion: the A that is present at position 47 of marker SEQ ID No:2 is missing in SEQ ID No:1. The marker allele SEQ ID No:3 differs from marker allele SEQ ID No:4 by a SNP on position 101, wherein SEQ ID No:3 has a G on position 101 and SEQ ID No:4 has an A on position 101. The marker allele SEQ ID No:5 differs from marker allele SEQ ID No:6 by a SNP on position 77, wherein SEQ ID No:5 has a T on position 77 and SEQ ID No:6 has a C on position 77. The marker allele SEQ ID No:7 differs from marker allele SEQ ID No:8 by a SNP on position 101, wherein SEQ ID No:7 has an A on position 101 and SEQ ID No:8 has a G on position 101. The marker allele SEQ ID No:9 differs from marker allele SEQ ID No:10 by a SNP on position 101, wherein SEQ ID No:9 has a T on position 101 and SEQ ID No:10 has a G on position 101. The marker allele SEQ ID No:11 differs from marker allele SEQ ID No:12 by a SNP on position 101, wherein SEQ ID No:11 has a T on position 101 and SEQ ID No:12 has a C on position 101. The marker allele SEQ ID No:13 differs from marker allele SEQ ID No:14 by a SNP on position 101, wherein SEQ ID No:13 has a C on position 101 and SEQ ID No:14 has a G on position 101. The marker allele SEQ ID No:15 differs from marker allele SEQ ID No:16 by a SNP on position 92, wherein SEQ ID No:15 has a T on position 92 and SEQ ID No:16 has a C on position 92.

The SNPs in these marker sequences may be used as molecular markers for detecting the presence of the QTL of the invention. These SNPs are the actual markers. When using a SNP as a marker the sequence surrounding the SNP is not necessarily of the same lengths as the sequences given here. The SNPs can for example be used for detecting the presence of the QTL of the invention in the progeny of a cross between an eggplant plant not comprising the QTL of the invention and an eggplant plant comprising the QTL of the invention, which plant may be a plant grown from a seed of which a representative sample was deposited with the NCIMB under NCIMB accession number 42507.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1: Development of the Plant of the Invention

The eggplant plant of the invention was obtained in the following way: in the selection of doubled haploid eggplant plants, that were generated starting with an F1 hybrid eggplant plant, 2 out of 41 plants were surprisingly found to produce seeds with the novel color of the invention. This was surprising as neither the F1 hybrid plants that were used as the basis for generating the doubled haploid population, nor F2 plants and subsequent generations resulting from self pollinations on said F1 hybrid plants had ever produced seeds with the novel color of the invention. The initially developed plant of the invention CB-2 was crossed with a wild type plant. Some further rounds of selfing, crossing and selection led to seeds of Solanum melongena comprising the QTL of the invention in a homozygous state.

These seeds were deposited with NCIMB Ltd, Ferguson Building, Craibstone Estate, Bucksburn, Aberdeen AB21 9YA, UK, on Jan. 5, 2016 under deposit accession numbers NCIMB 42507. Seeds of this deposit comprise the QTL in a homozygous state.

### Example 2: Phenotypic Characterization of Plants of the Invention

Plants of the invention carrying the genetic determinant homozygously may be identified by the novel color of their seeds.

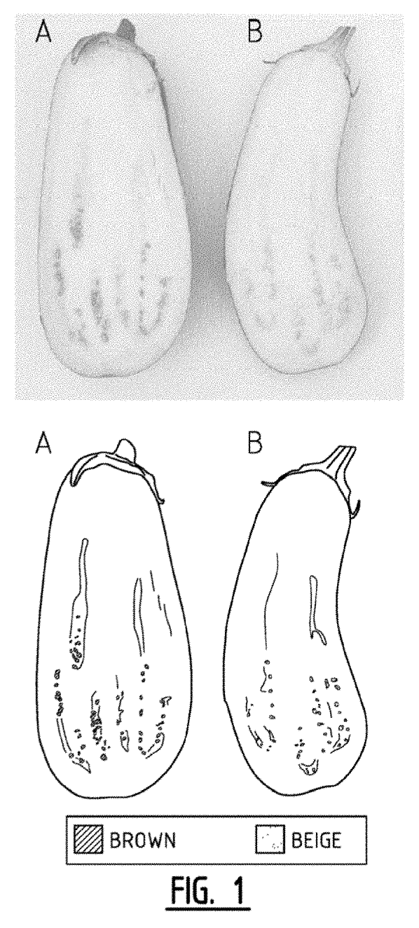

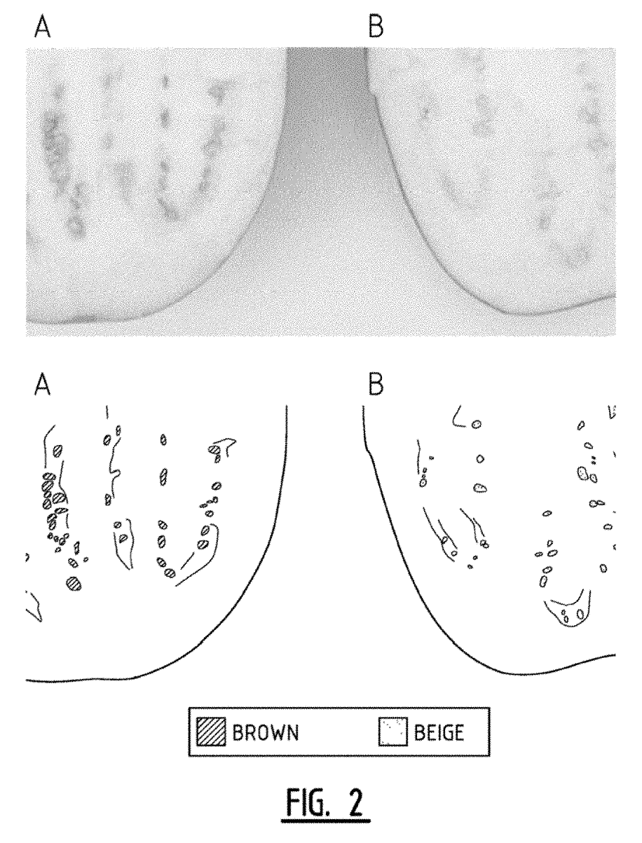

The color of the mature seeds of plants of the invention carrying the QTL of the invention homozygously is much lighter than that of isogenic plants carrying the QTL of the invention either heterozygously or not at all (FIGS. 1 and 2). This lighter color of mature seeds of plants carrying the genetic determinant homozygously, as compared to isogenic plants carrying the genetic determinant either heterozygously or not at all, was most apparent in fresh mature seeds immediately after harvest of the fruit. Mature seeds are as those present in the fully physiologically developed eggplant fruit. Fresh seeds are as those present in the eggplant fruit immediately after harvest or those that have been taken from the fruit very recently and are still wet. While the color of fresh mature wild type eggplant seeds not carrying the QTL of the invention could be called middle brown, sandy brown, bronze, or copper, the color of fresh mature seeds of plants of the invention carrying the genetic determinant homozygously could be indicated as off-white, creamy white, beige, very light yellow or light grayish yellow.

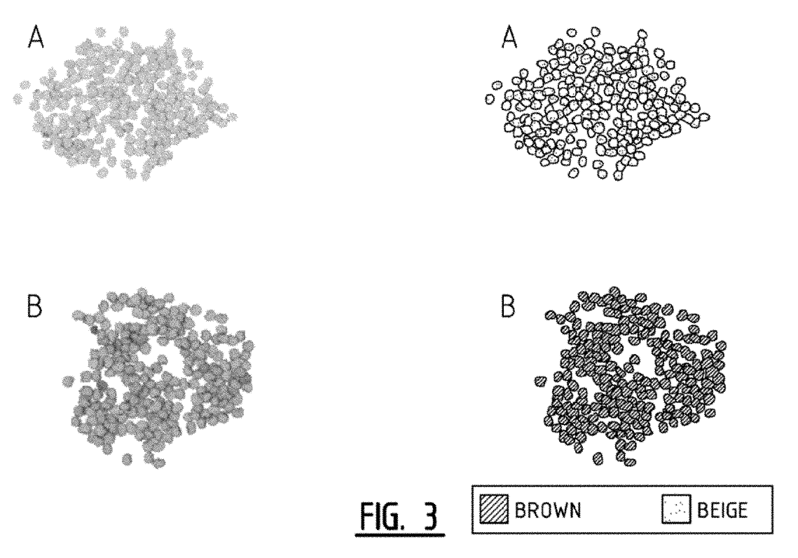

Upon drying of the seeds the color of both wild type seeds and seeds of plants of the invention changed. However, also when completely dry the color of mature seeds of plants of the invention was clearly distinguishable from and lighter than that of mature wild type seeds (FIG. 3). While the color of dried mature wild type eggplant seeds not carrying the QTL of the invention could be called brown, sandy brown, middle brown, bronze or copper, the color of dried mature seeds of plants of the invention carrying the genetic determinant homozygously could be indicated as beige, light yellow, pale yellow, buff or khaki.

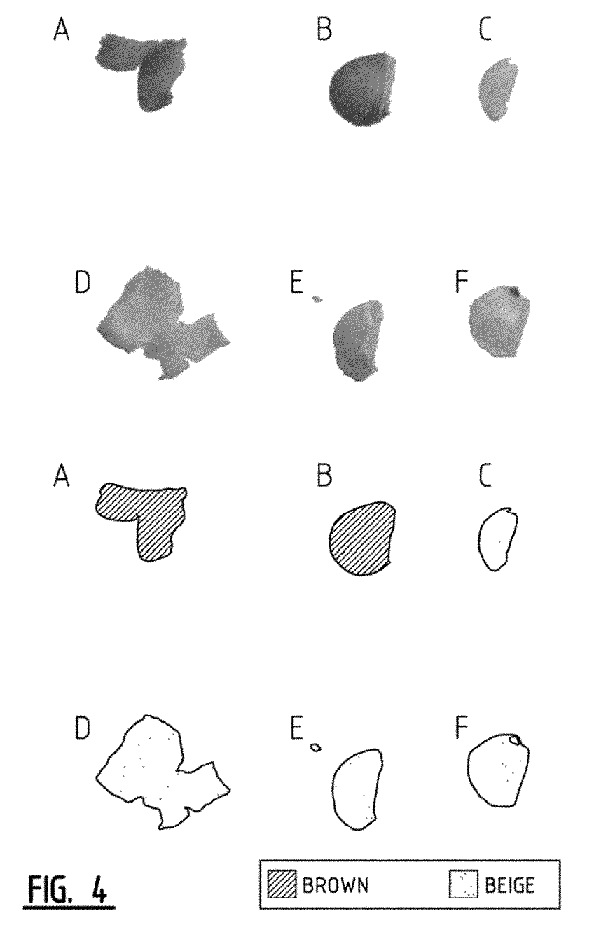

When the seed coat was peeled off of both mature seeds of plants showing the novel seed color phenotype and wild type control seeds it became clear that the novel seed color phenotype of seeds of the invention is due to the reduction or absence of brown pigments in the seed coat, also called testa, revealing the color of the cotyledons beneath the seed coat (FIG. 4).

The coloration of the seeds of the invention and wild type seeds was scored using the RHS color chart (The Royal Horticultural Society, London, UK). The color of fresh mature wild type seeds not carrying the QTL of the invention was scored 165B at its darkest parts and 165C at its lightest parts, while the color of fresh mature seeds of the invention was scored 161D at its lightest parts and 161C at its darkest parts (Table 2). The color of dried mature wild type seeds was scored 162A, while the color of dried mature seeds of the invention was scored 164B (Table 2).

TABLE 2
RHS color estimation of fresh and dried wild type eggplant seeds and
seeds of an eggplant of the invention carrying the QTL of the invention
homozygously. The RHS color codes were also converted into CIELAB
L*a*b* color values (D65 illuminant and a 10 degree angle of observer).

| | RHS color fresh | | | RHS color dried | | |
|---|---|---|---|---|---|---|
| | mature seeds | | | mature seeds | | |
| Genotype | L* | a* | b* | L* | a* | b* |
| | 161D | | | 162A | | |
| Deposit NCIMB 42507 | 73.65 | 0.21 | 25.42 | 59.18 | 9.85 | 32.89 |
| (carrying the QTL | | | | | | |
| homozygously | | | | | | |
| | 161C | | | | | |
| | 67.68 | 8.54 | 27.91 | | | |
| | 165B | | | 164B | | |
| Wild type seeds | 32.93 | 20.53 | 18.32 | 46.33 | 19.13 | 25.30 |

| 165C | | |
|---|---|---|
| 48.68 | 19.37 | 24.61 |

Environmental conditions, including light source, object size, color background, and angle of vision or illumination may affect how a colored object appears to a human observer. In order to overcome this subjectivity, color measurement systems and instruments have been developed for quantifying colors and expressing colors in terms of variables which describe the color. Besides estimating seed color using an RHS color chart as described above, two experimental methodologies were used: colorimetry/spectophotometry and image analysis.

A Konica Minolta hand-held colorimeter model CM700d with 3 mm measurement aperture was used in De Lier, The Netherlands, to score the color of mature eggplant seeds, using CIELAB L*a*b* color space, D65 illuminant and a 10 degree angle of observer. The instrument was calibrated prior to use according to the manufacturer's instructions. For the colorimeter measurements about 10 seeds were piled up on a white background, covering the background with seeds as well as possible. The mature wild type seeds and seeds of the invention were of a similar size. Colorimeter scores were taken four times on each sample. Average values per sample are presented in Table 3.

TABLE 3
Average color of fresh and dried wild type eggplant seeds and seeds of an
eggplant of the invention carrying the QTL of the invention homozygously
as determined by a Konica Minolta colorimeter CM700d using CIELAB
L*a*b* color space, D65 illuminant and a 10 degree angle of observer.

| | Average color | | | Average color | | |
|---|---|---|---|---|---|---|
| | fresh mature | | | dried mature | | |
| | seeds (10°/D65) | | | seeds (10°/D65) | | |
| Genotype | L* | a* | b* | L* | a* | b* |
| Deposit NCIMB 42507 | 90.10 | 1.64 | −6.49 | 63.07 | 9.47 | 36.28 |
| (carrying the QTL | | | | | | |
| homozygously) | | | | | | |
| Wild type seeds | 84.64 | 4.02 | −1.24 | 61.31 | 9.67 | 18.52 |

Photography was conducted in a standardized set-up in a darkened room using a Nikon D7000 camera with a Nikon AF-S 35 mm f/1.8G DX 35 mm lens with circular B+W polarisation filter. The standardized camera set up used daylight fluorescent lamps (4×36 watts, 5400 K, CRI 98, 40 kHz) with a polarisation filter. Lamp heads were angled at 45 degrees to the sample platform. Prior to taking photographs, lamps were turned on and allowed to warm up for at least 30 minutes. The camera was mounted on a stand with the lens pointing down and positioned over the sample platform. About 10 mature seeds that were clearly separated from each other were photographed for each sample.

Color correction of the photographs was performed using an ImageJ macro (1.48u) and the X-rite colorchecker passport colorchart. Image analysis and generation of calibrated RGB values was performed using a CellProfiler pipeline. The calibrated RGB color values were then translated into CIELAB L*a*b* color values (D65 illuminant and a 10 degree angle of observer) using a color calibration algorithm.

TABLE 4
Average color in calibrated RGB values and in calibrated CIE L*a*b*
values (10°/D65) of fresh and dried wild type eggplant seeds and
seeds of an eggplant of the invention carrying the QTL of the invention
homozygously as determined by image analysis.

| | Average calibrated color | | | Average calibrated color | | |
|---|---|---|---|---|---|---|
| Genotype | fresh mature seeds | | | dried mature seeds | | |
| Deposit NCIMB | R | G | B | R | G | B |
| 42507 (carrying | 166 | 168 | 163 | 184 | 147 | 62 |
| the QTL | L* | a* | b* | L* | a* | b* |
| homozygously) | 68.50 | −1.03 | 1.11 | 62.80 | 4.72 | 48.40 |
| Wild type seeds | R | G | B | R | G | B |
| | 151 | 150 | 130 | 136 | 132 | 104 |
| | L* | a* | b* | L* | a* | b* |
| | 61.70 | −3.27 | 10.41 | 54.80 | −3.33 | 15.19 |

The CIELAB color scale is used widely to measure color and demonstrate differences in color. The scale includes 3 data variables: L*, a* and b*. L* indicates lightness on a 0 to 100 scale, where 0 is black and 100 is white. The variables a* and b* indicate the amount of red, green, blue and yellow color: a* value indicates color change from green (negative values) to red (positive values), while b* indicates color change from yellow (positive values) to blue (negative values). Differences in color between two samples can be expressed in terms of change in L* and/or a*, and/or b*.

As is clear from the different L*a*b* scores in Table 2 to 4, color parameters vary dependent on the method used for quantifying colors. Still, when comparing the L* (10°/D65) scores for a particular color quantification method (either color estimation using an RHS color chart, colorimetry or image analysis) of wild type mature fresh seed with fresh mature seed of the invention, the L* score is always higher for the seed of the invention, when compared to the wild type seed not carrying the QTL of the invention. This is the same when comparing the L* (10°/D65) scores for a particular color quantification method (either color estimation using an RHS color chart, colorimetry or image analysis) of wild type dried mature seed with dried mature seed of the invention, the L* score is always higher for the seed of the invention, when compared to the wild type seed not carrying the QTL of the invention.

### Example 3: QTL Analysis on Trait of the Invention

Plants of the invention carrying the genetic determinant homozygously may be identified by the novel color of their seeds.

To map the trait of the invention and identify a QTL for the trait of the invention doubled haploid (DH) line CB-2 which carried the genetic determinant homozygously was crossed with DH line BB-15 which produced normally colored seeds and did not carry the genetic determinant of the invention. The F1 plants resulting from this cross were used to generate the GH population, made up of 371 DH lines. These 371 DH plants were grown and phenotyped for the color of the seeds they produced.

All the DH plants from the GH population were scored with two genotypes: AA for homozygous parent CB-2 and BB for homozygous parent BB-15. After filtering out non-polymorphic markers, markers with strong high missing values as well as strong distortion, and performing marker phase correction, genetic map construction of the remaining 91 SNP markers was done in JoinMap 4.0 software (Kyazma B.V., Wageningen). The maximum likelihood mapping approach was first used to estimate the order of the markers in a linkage group. This was followed by regression mapping to predict the position of markers in linkage groups using the marker start orders obtained from maximum likelihood mapping. Haldane mapping function was used to convert recombination frequency between markers into genetic distance between markers (in centiMorgan, cM). Linkage group numbering was in accordance with the Draft Genome Sequence of Eggplant (Solanum melongena L.) (Hirakawa et al., Draft Genome Sequence of Eggplant (Solanum melongena L.): the Representative Solanum Species Indigenous to the Old World, 2014, DNA Research 21, 649-660). Orientation of markers on the map was adjusted using internal reference map positions.

A histogram was constructed for the trait of the invention to see the data distribution: the trait of the invention had equal distribution of two scores: novel seed color and normal seed color.

QTL mapping of the trait of the invention was carried out in the GH population. A step-wise regression approach was used for QTL mapping using R statistical software. One major QTL for the trait of the invention was detected on Linkage Group 11 with 71% explained variance. 16 Markers were mapped on Linkage Group 11, which had a total length of 63.4 cM. Marker SEQ ID No:13 at 36.7 cM on Linkage Group 11 was identified as a peak marker. The LOD score at the peak marker was 193.6, the explained variance 71% and the additive effect −0.48. The upper flanking marker is SEQ ID No:9 at 34.4 cM and the lower flanking marker SEQ ID No:15 at 40.8 cM. Additional markers are SEQ ID No:7, SEQ ID No:5, SEQ ID No:3 and SEQ ID No:1 at 34.4 cM and marker SEQ ID No:11 at 36.7 cM.

### Example 4: Transfer of the Trait of the Invention to Other Eggplants

Eggplant plants of the invention carrying the QTL of the invention homozygously (donor parent; grown from seeds of the deposit, NCIMB 42507) were crossed with several different wild type eggplant plants, which did not carry the trait of the invention, but differed in type, fruit form and fruit color. In case the eggplant plant of the invention was used as a mother in the cross and the wild type plant as a pollen donor the F1 seeds resulting from this cross had the novel seed color of the invention. In case the wild type plant was chosen as the mother in the cross and the plant of the invention as the pollen donor, however, the F1 seeds resulting from this cross had a normal seed color. The color of the seeds thus is determined by the genotype of the plant that produces the seeds (the mother plant in a cross).

Regardless of whether the eggplant plant of the invention was used as the mother or as the pollen donor in a cross with a wild type plant, plants grown from the resulting F1 seeds had the same phenotype as the wild type plant, i.e. they produced seeds of a normal color, suggesting that the trait is recessive. Nevertheless, the presence of the trait of the invention in a heterozygous state could be detected in all F1 plants by means of molecular markers. The Single Nucleotide Polymorphism (SNP) markers that were used for this purpose are presented in Table 1 as SEQ ID No:1, SEQ ID No:3, SEQ ID No:5, SEQ ID No:7, SEQ ID No:9, SEQ ID No:11, SEQ ID No:13 and SEQ ID No:15.

In the F2 generation the trait of the invention segregated in a manner that corresponds with a monogenic recessive inheritance (Table 5).

TABLE 5
Segregations of the trait of the invention in an F2 population from
a cross of a plant of the invention with a WT eggplant parent
(with normally colored seeds).

| | WT seed | | | Chi-square | |
|---|---|---|---|---|---|
| | color | Novel seed color | Total | probability | p > 0.05? |
| observed | 22 | 8 | 30 | 0.83 | yes |
| expected | 22.5 | 7.5 | 30 | | |

Chi-square tests confirmed that the observed numbers of F2 plants that had the wild type parental phenotype (normal seed color) and the donor parent phenotype (novel seed color) were in agreement with what is expected if a trait segregates in monogenic recessive fashion, namely 3:1 (WT parent phenotype:donor parent phenotype). The chi-square probability value is above 0.05. From the segregation data in Table 5 it can thus be concluded that the QTL of the invention behaves as a monogenic recessive trait.

The invention is further described by the following numbered paragraphs:

1. An eggplant plant (Solanum melongena L.) comprising a QTL which when homozygously present leads to the eggplant plant producing seeds with a novel color, wherein said QTL is the same as a QTL that is present in the genome of a plant grown from a seed of deposit number NCIMB 42507 and is linked therein to at least one marker selected from the group consisting of SEQ ID No:1, SEQ ID No:3, SEQ ID No:5, SEQ ID No:7, SEQ ID No:9, SEQ ID No:11, SEQ ID No:13 and SEQ ID No:15.

2. An eggplant plant of paragraph 1, wherein the QTL is as present in the genome of, or obtainable from, an eggplant plant grown from a seed of which a representative sample was deposited at the NCIMB under accession number NCIMB 42507.

3. An eggplant plant of paragraph 1 or 2, wherein the novel seed color is defined as fresh seeds produced by said plant having a lighter color at the mature seed stage, as compared to seeds produced by a plant not comprising the QTL homozygously.

4. An eggplant plant of any one of the paragraphs 1-3, wherein the novel seed color is defined as dried seeds produced by said plants having a lighter color at the mature seed stage, as compared to seeds produced by a plant not comprising the QTL homozygously.

5. An eggplant plant of any of the paragraphs 1-4, wherein the seed coats of the mature seeds produced by said plant have a reduced amount of brown pigments, as compared to the seed coats of mature seeds produced by a plant not comprising the QTL homozygously.

6. An eggplant plant of any of the paragraphs 1-5, wherein the QTL is homozygously present.

7. Seed of an eggplant plant as defined in any of the paragraphs 1-6, comprising the QTL as defined in paragraph 1 or 2, which QTL is preferably homozygously present.

8. Seed capable of growing into an eggplant plant of any of the paragraphs 1-6, comprising the QTL as defined in paragraph 1 or 2, which QTL is preferably homozygously present.

9. Progeny of an eggplant plant of any of the paragraphs 1-4 or progeny of an eggplant plant grown from seed of paragraph 5 or 6, wherein the progeny of the plant comprises the QTL as defined in paragraph 1 or 2.

10. Propagation material derived from an eggplant plant of any one of the paragraphs 1-6 or 9 or derived from an eggplant seed of paragraph 5 or 6, wherein the propagation material comprises the QTL as defined in paragraph 1 or 2, which QTL is preferably homozygously present.

11. Propagation material capable of growing into an eggplant plant of any one of the paragraphs 1-6 or 9, comprising the QTL as defined in paragraph 1 or 2, which QTL is preferably homozygously present.

12. Propagation material of paragraph 8 or 9, wherein the propagation material is selected from the group consisting of callus, a microspore, pollen, an ovary, an ovule, an embryo, an embryo sac, an egg cell, a cutting, a root, a stem, a cell, a protoplast, a leaf, a cotyledon, a hypocotyl, a meristematic cell, a root, a root tip, a microspore, an anther, a flower, a seed and a stem, or is a part or tissue culture thereof.

13. An eggplant fruit, or a part thereof, or a food product made of a fruit or of a part thereof, or a processed food product made thereof, harvested from an eggplant of any one of the paragraphs 1-6 or 9, comprising the QTL as defined in paragraph 1 or 2, wherein the QTL is preferably homozygously present.

14. Use of an eggplant plant of any one of the paragraphs 1-6 or 9 as germplasm in a breeding program for the development of eggplant plants comprising a QTL that leads to said plants producing seeds with a novel seed color as defined in paragraph 3, 4 or 5.

15. A nucleic acid or a part thereof, optionally in isolated form, which causes a novel seed color, which nucleic acid originates from an eggplant plant of any of the paragraphs 1-6 or 9, and is linked therein to at least one marker selected from the group consisting of SEQ ID No:1, SEQ ID No:3, SEQ ID No:5, SEQ ID No:7, SEQ ID No:9, SEQ ID No:11, SEQ ID No:13 and SEQ ID No:15.

16. Use of a molecular marker selected from the group consisting of SEQ ID No:1, SEQ ID No:3, SEQ ID No:5, SEQ ID No:7, SEQ ID No:9, SEQ ID No:11, SEQ ID No:13 and SEQ ID No:15 to select and/or develop eggplant plants producing seeds with a novel color, in particular as in paragraph 3, 4 or 5.

17. Use of a molecular marker selected from the group consisting of SEQ ID No:1, SEQ ID No:3, SEQ ID No:5, SEQ ID No:7, SEQ ID No:9, SEQ ID No:11, SEQ ID No:13 and SEQ ID No:15 to identify the QTL of paragraph 1 or 2 in an eggplant plant of any one of paragraphs 1-6 or 9.

18. Use of a molecular marker selected from the group consisting of SEQ ID No:1, SEQ ID No:3, SEQ ID No:5, SEQ ID No:7, SEQ ID No:9, SEQ ID No:11, SEQ ID No:13 and SEQ ID No:15 to develop other markers linked to the QTL as in paragraph 1 or 2.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. An eggplant plant (Solanum melongena L.) comprising a QTL which when homozygously present leads to the eggplant plant producing seeds with a novel color, wherein said QTL is the same as a QTL that is present in the genome of a plant grown from a seed of deposit number NCIMB 42507 and is linked therein to at least one marker selected from the group consisting of SEQ ID No:1, SEQ ID No:3, SEQ ID No:5, SEQ ID No:7, SEQ ID No:9, SEQ ID No:11, SEQ ID No:13 and SEQ ID No:15.

2. The eggplant plant as claimed in claim 1, wherein the QTL is as present in the genome of, or obtainable from, an eggplant plant grown from a seed of which a representative sample was deposited at the NCIMB under accession number NCIMB 42507.

3. The eggplant plant as claimed in claim 1, wherein the novel seed color is defined as fresh seeds produced by said plant having a lighter color at the mature seed stage, as compared to seeds produced by a plant not comprising the QTL homozygously.

4. The eggplant plant as claimed in claim 1, wherein the novel seed color is defined as dried seeds produced by said plants having a lighter color at the mature seed stage, as compared to seeds produced by a plant not comprising the QTL homozygously.

5. The eggplant plant as claimed in claim 1, wherein the seed coats of the mature seeds produced by said plant have a reduced amount of brown pigments, as compared to the seed coats of mature seeds produced by a plant not comprising the QTL homozygously.

6. The eggplant plant as claimed in claim 1, wherein the QTL is homozygously present.

7. A seed of the eggplant plant of claim 1, comprising a QTL that is present in the genome of a plant grown from a seed of deposit number NCIMB 42507 and is linked therein to at least one marker selected from the group consisting of SEQ ID No:1, SEQ ID No:3, SEQ ID No:5, SEQ ID No:7, SEQ ID No:9, SEQ ID No:11, SEQ ID No:13 and SEQ ID No:15, which QTL is preferably homozygously present.

8. A seed capable of growing into an eggplant plant as claimed in claim 1, comprising a QTL that is present in the genome of a plant grown from a seed of deposit number NCIMB 42507 and is linked therein to at least one marker selected from the group consisting of SEQ ID No:1, SEQ ID No:3, SEQ ID No:5, SEQ ID No:7, SEQ ID No:9, SEQ ID No:11, SEQ ID No:13 and SEQ ID No:15 which QTL is preferably homozygously present.

9. A progeny of an eggplant plant as claimed in claim 1 or a progeny of an eggplant plant grown from the seed as claimed in claim 7 or 8, wherein the progeny of the plant comprises a QTL that is present in the genome of a plant grown from a seed of deposit number NCIMB 42507 and is linked therein to at least one marker selected from the group consisting of SEQ ID No:1, SEQ ID No:3, SEQ ID No:5, SEQ ID No:7, SEQ ID No:9, SEQ ID No:11, SEQ ID No:13 and SEQ ID No:15.

10. A propagation material derived from the eggplant plant as claimed in claim 1 or derived from the eggplant seed as claimed in claim 7 or 8, wherein the propagation material comprises a QTL that is present in the genome of a plant grown from a seed of deposit number NCIMB 42507 and is linked therein to at least one marker selected from the group consisting of SEQ ID No:1, SEQ ID No:3, SEQ ID No:5, SEQ ID No:7, SEQ ID No:9, SEQ ID No:11, SEQ ID No:13 and SEQ ID No:15, which QTL is preferably homozygously present.

11. A propagation material capable of growing into an eggplant plant as claimed in any claim 1, comprising a QTL that is present in the genome of a plant grown from a seed of deposit number NCIMB 42507 and is linked therein to at least one marker selected from the group consisting of SEQ ID No:1, SEQ ID No:3, SEQ ID No:5, SEQ ID No:7, SEQ ID No:9, SEQ ID No:11, SEQ ID No:13 and SEQ ID No:15, which QTL is preferably homozygously present.

12. A propagation material capable of growing into an eggplant plant as claimed in any claim 9, comprising a QTL that is present in the genome of a plant grown from a seed of deposit number NCIMB 42507 and is linked therein to at least one marker selected from the group consisting of SEQ ID No:1, SEQ ID No:3, SEQ ID No:5, SEQ ID No:7, SEQ ID No:9, SEQ ID No:11, SEQ ID No:13 and SEQ ID No:15, which QTL is preferably homozygously present.

13. The propagation material as claimed in claim 10, wherein the propagation material is selected from the group consisting of callus, a microspore, pollen, an ovary, an ovule, an embryo, an embryo sac, an egg cell, a cutting, a root, a stem, a cell, a protoplast, a leaf, a cotyledon, a hypocotyl, a meristematic cell, a root, a root tip, a microspore, an anther, a flower, a seed and a stem, or is a part or tissue culture thereof.

14. The propagation material as claimed in claim 11, wherein the propagation material is selected from the group consisting of callus, a microspore, pollen, an ovary, an ovule, an embryo, an embryo sac, an egg cell, a cutting, a root, a stem, a cell, a protoplast, a leaf, a cotyledon, a hypocotyl, a meristematic cell, a root, a root tip, a microspore, an anther, a flower, a seed and a stem, or is a part or tissue culture thereof.

15. The propagation material as claimed in claim 12, wherein the propagation material is selected from the group consisting of callus, a microspore, pollen, an ovary, an ovule, an embryo, an embryo sac, an egg cell, a cutting, a root, a stem, a cell, a protoplast, a leaf, a cotyledon, a hypocotyl, a meristematic cell, a root, a root tip, a microspore, an anther, a flower, a seed and a stem, or is a part or tissue culture thereof.

16. An eggplant fruit, or a part thereof, or a food product made of a fruit or of a part thereof, or a processed food product made thereof, harvested from an eggplant as claimed in claim 1, comprising a QTL that is present in the genome of a plant grown from a seed of deposit number NCIMB 42507 and is linked therein to at least one marker selected from the group consisting of SEQ ID No:1, SEQ ID No:3, SEQ ID No:5, SEQ ID No:7, SEQ ID No:9, SEQ ID No:11, SEQ ID No:13 and SEQ ID No:15, wherein the QTL is preferably homozygously present.

17. An eggplant fruit, or a part thereof, or a food product made of a fruit or of a part thereof, or a processed food product made thereof, harvested from an eggplant as claimed in claim 9, comprising a QTL that is present in the genome of a plant grown from a seed of deposit number NCIMB 42507 and is linked therein to at least one marker selected from the group consisting of SEQ ID No:1, SEQ ID No:3, SEQ ID No:5, SEQ ID No:7, SEQ ID No:9, SEQ ID No:11, SEQ ID No:13 and SEQ ID No:15, wherein the QTL is preferably homozygously present.

18. A nucleic acid or a part thereof, optionally in isolated form, which causes a novel seed color, which nucleic acid originates from an eggplant plant as claimed in claim 1, and is linked therein to at least one marker selected from the group consisting of SEQ ID No:1, SEQ ID No:3, SEQ ID No:5, SEQ ID No:7, SEQ ID No:9, SEQ ID No:11, SEQ ID No:13 and SEQ ID No:15.

19. A nucleic acid or a part thereof, optionally in isolated form, which causes a novel seed color, which nucleic acid originates from an eggplant plant as claimed in claim 9, and is linked therein to at least one marker selected from the group consisting of SEQ ID No:1, SEQ ID No:3, SEQ ID No:5, SEQ ID No:7, SEQ ID No:9, SEQ ID No:11, SEQ ID No:13 and SEQ ID No:15.


SEQUENCE LISTING (16 sequences)

SEQ ID NO: 1 - DNA, 146 bp, Solanum melongena
  source 1..146: /organism=“Solanum melongena” /mol_type=“unassigned DNA”
ttttttagacatttgcatcaactttgcagacgatacttaggaatcacataagatgttgaactctagctgttactaaaccgaatttctcagtcatgtcaatatccttcggtgtcatgccatttggcaactcccagtcaaagcaatga

SEQ ID NO: 2 - DNA, 147 bp, Solanum melongena
  source 1..147: /organism=“Solanum melongena” /mol_type=“unassigned DNA”
ttttttagacatttgcatcaactttgcagacgatacttaggaatcaacataagatgttgaactctagctgttactaaaccgaatttctcagtcatgtcaatatccttcggtgtcatgccatttggcaactcccagtcaaagcaatga

SEQ ID NO: 3 - DNA, 177 bp, Solanum melongena
  source 1..177: /organism=“Solanum melongena” /mol_type=“unassigned DNA”
ccttcggtgtcatgccatttggcaactcccagtcaaagcaatgaaccaattgtgcgagcaccaaacgaaccgtggtgagccccaactgcaaacccgggcagcttcttctgcccgaaccaaatggtaaaagctcaaaatgtcgcccgtgaaggtcaatgttgcttccttgaaatctct

SEQ ID NO: 4 - DNA, 177 bp, Solanum melongena
  source 1..177: /organism=“Solanum melongena” /mol_type=“unassigned DNA”
ccttcggtgtcatgccatttggcaactcccagtcaaagcaatgaaccaattgtgcgagcaccaaacgaaccgtggtgagccccaactgcaaacccgggcaacttcttctgcccgaaccaaatggtaaaagctcaaaatgtcgcccgtgaaggtcaatgttgcttccttgaaatctct

SEQ ID NO: 5 - DNA, 177 bp, Solanum melongena
  source 1..177: /organism=“Solanum melongena” /mol_type=“unassigned DNA”
cttcttctgcccgaaccaaatggtaaaagctcaaaatgtcgcccgtgaaggtcaatgttgcttccttgaaatctcttaggtttgaacttttctggctcaacccaaatgtctggatctcttcctattgtccaaacattgatgagaagtcgggatcctttgggtatgtgaaaaccatca

SEQ ID NO: 6 - DNA, 177 bp, Solanum melongena
  source 1..177: /organism=“Solanum melongena” /mol_type=“unassigned DNA”
cttcttctgcccgaaccaaatggtaaaagctcaaaatgtcgcccgtgaaggtcaatgttgcttccttgaaatctctcaggtttgaacttttctggctcaacccaaatgtctggatctcttcctattgtccaaacattgatgagaagtcgggatcctttgggtatgtgaaaaccatca

SEQ ID NO: 7 - DNA, 201 bp, Solanum melongena
  source 1..201: /organism=“Solanum melongena” /mol_type=“unassigned DNA”
cgactcgtggggaatcaacaagggtgcaacaggatggagtctactactttcttttatgaccatatctaaatattcaagattttctaagtctgattcttctaccattctgtttttgccagctacttgttccaactctttttgaagccttttcatcacattggggtgccttagaagttctgctaaaatccattctattgttgt

SEQ ID NO: 8 - DNA, 201 bp, Solanum melongena
  source 1..201: /organism=“Solanum melongena” /mol_type=“unassigned DNA”
cgactcgtggggaatcaacaagggtgcaacaggatggagtctactactttcttttatgaccatatctaaatattcaagattttctaagtctgattcttctgccattctgtttttgccagctacttgttccaactctttttgaagccttttcatcacattggggtgccttagaagttctgctaaaatccattctattgttgt

SEQ ID NO: 9 - DNA, 201 bp, Solanum melongena
  source 1..201: /organism=“Solanum melongena” /mol_type=“unassigned DNA”
taaactagggaggttctgccggcctgcaattttcaagttccatggctcctcagaaccaccaccaacttccttgagtttctctaatgatcacttgctcacatcgaactcgagatctctgtctgggcaaattgagtctggtacagaaattgttgagcatgttgttccagagggagttactgcagtttcagcatctacacatct

SEQ ID NO: 10 - DNA, 201 bp, Solanum melongena
  source 1..201: /organism=“Solanum melongena” /mol_type=“unassigned DNA”
taaactagggaggttctgccggcctgcaattttcaagttccatggctcctcagaaccaccaccaacttccttgagtttctctaatgatcacttgctcacagcgaactcgagatctctgtctgggcaaattgagtctggtacagaaattgttgagcatgttgttccagagggagttactgcagtttcagcatctacacatct

SEQ ID NO: 11 - DNA, 201 bp, Solanum melongena
  source 1..201: /organism=“Solanum melongena” /mol_type=“unassigned DNA”
ctccggaaacaacctctttatctctgtgaggtagtgataaggtctgcgtacatttcaccctccccagattccactgggtatgttgttgttgttcatgtattatactataagcttcaataaatcgaaaatttgttcattacattccttttcttgtttatttttgacagacaaggagaagcatcttcagctacttcaactgct

SEQ ID NO: 12 - DNA, 201 bp, Solanum melongena
  source 1..201: /organism=“Solanum melongena” /mol_type=“unassigned DNA”
ctccggaaacaacctctttatctctgtgaggtagtgataaggtctgcgtacatttcaccctccccagattccactgggtatgttgttgttgttcatgtatcatactataagcttcaataaatcgaaaatttgttcattacattccttttcttgtttatttttgacagacaaggagaagcatcttcagctacttcaactgct

SEQ ID NO: 13 - DNA, 201 bp, Solanum melongena
  source 1..201: /organism=“Solanum melongena” /mol_type=“unassigned DNA”
cgaactgtgacatcaagcactgtttgtaatgcttcaatctgcaaactggaatcgtgacaagcagacaagctaggcttgttttataagctcagccaaattacagagagtagttgaaatatctgaaactcaagcagccatggggttcggttcttgatctttttgtaccagagttcaacgacaaatacaaacttttgctcacta

SEQ ID NO: 14 - DNA, 201 bp, Solanum melongena
  source 1..201: /organism=“Solanum melongena” /mol_type=“unassigned DNA”
cgaactgtgacatcaagcactgtttgtaatgcttcaatctgcaaactggaatcgtgacaagcagacaagctaggcttgttttataagctcagccaaattagagagagtagttgaaatatctgaaactcaagcagccatggggttcggttcttgatctttttgtaccagagttcaacgacaaatacaaacttttgctcacta

SEQ ID NO: 15 - DNA, 192 bp, Solanum melongena
  source 1..192: /organism=“Solanum melongena” /mol_type=“unassigned DNA”
aaccaacaccaaaaccacctgttcatagcaaaccaccaccacctggttggaagtcttcgggttcgcataataagcgatctccaccgccacctatatcgactcctctaccttcaccaccaccaccaacttacaaaatctctcctacgcctagccctccatcttcttcttcttctcccccaccacctgtttaca

SEQ ID NO: 16 - DNA, 192 bp, Solanum melongena
  source 1..192: /organism=“Solanum melongena” /mol_type=“unassigned DNA”
aaccaacaccaaaaccacctgttcatagcaaaccaccaccacctggttggaagtcttcgggttcgcataataagcgatctccaccgccacccatatcgactcctctaccttcaccaccaccaccaacttacaaaatctctcctacgcctagccctccatcttcttcttcttctcccccaccacctgtttaca